(19) United States
(12) Patent Application Publication
(10) Pub. No.: US 2020/0152326 A1
(43) Pub. Date: May 14, 2020

(54) BLOOD PATHOLOGY IMAGE ANALYSIS AND DIAGNOSIS USING MACHINE LEARNING AND DATA ANALYTICS

(71) Applicant: International Business Machines Corporation, Armonk, NY (US)

(72) Inventors: Marta Sanchez-Martin, Somerville, MA (US); Claudia S. Huettner, Jamaica Plain, MA (US); Jia Xu, Somerville, MA (US); Cheryl Eifert, Watertown, MA (US); Elinor Dehan, Haniel (IL); Shang Xue, Cambridge, MA (US); Vanessa Michelini, Boca Raton, FL (US)

(21) Appl. No.: 16/185,701

(22) Filed: Nov. 9, 2018

(51) Int. Cl. G16H 50/20; G06N 99/00

(52) U.S. Cl. CPC G16H 50/20; G06N 99/005

(57) ABSTRACT

Methods, systems, and computer readable media are provided for processing microscopic images of a biological sample from a patient. One or more images of a blood sample from a microscope is obtained, each image comprising a plurality of different types of cells. The one or more images are processed by a machine learning system to classify individual cells into one of a plurality of cell categories. The cells in each cell category are analyzed to determine characteristics of the respective cell category. A diagnosis or list of possible diagnosis are determined based on the classification and characteristics of the cells for the patient in an automated manner.

### TECHNICAL FIELD

Present invention embodiments relate to automated techniques for analysis of biological samples, and more specifically, to machine learning techniques and data analytics of images of blood samples for diagnosis of a disease.

### DISCUSSION OF THE RELATED ART

Blood diseases are typically diagnosed by a combination of immunophenotyping and pathology imaging techniques such as microscopy. Immunophenotyping may involve applying various stains or other reagents to a biological sample, such as a bone marrow, blood or other tissue sample or any other suitable biological sample comprising a heterogeneous population of cells, in order to identify the presence of particular types of cells within the population of cells. Microscopy imaging, immunohistochemistry (IHC) or direct observation of a blood smear sample may also be performed as part of the diagnostic process. In some cases, the biological sample may be incubated under suitable conditions with an antibody that binds to a cell surface antigen to identify the cell. The antibodies may be fluorophore or fluorochrome conjugated, and the presence of the fluorophore or fluorochrome may be detected by microscopy or by flow cytometry.

Often, physicians or other medical personnel conduct and interpret the results of immunophenotyping and IHC assays, which typically involve generation of a series of images of the biological sample, a process that is both manually intensive and time consuming. However, manual review is slow and subject to pathologist subjectivity, and different pathologists may reach different conclusions when analyzing the same slide. This may lead to delays in diagnosis and in appropriate patient care, especially if the patient is misdiagnosed.

In recent years, advances in laboratory automation have allowed certain laboratory-based tasks to become more automated. However, these automated techniques are often limited in scope and are not standardized. Hospitals, clinics, and other laboratories which analyze biological samples for diagnosing blood cancers or other diseases do not typically use automated methods, instead relying on clinician judgement and pathologist analysis, a process that may be both time consuming and subject to human error.

### SUMMARY

According to embodiments of the present invention, computer-implemented methods, systems, and computer readable media are provided for analyzing microscopic images of a biological sample using machine learning techniques and data analytics to generate a diagnosis for a patient. In some cases, the biological sample may be a blood sample. The blood sample may be processed in a manner to allow visualization of the cells and/or other components of the blood sample. One or more images of the blood sample are obtained, each image comprising a plurality of different types of cells. The one or more images are analyzed with a machine learning system to quantify and classify individual cells into one of a plurality of cell categories. The cells in each cell category are analyzed to determine characteristics of the respective cell category. A diagnosis is determined based on the quantity, the classification, and characteristics of the cells to determine a diagnosis for the patient. Unlike traditional manners of analyzing blood samples, which rely on one or more pathologists to manually interpret the slides, present techniques provide for analyzing cells and generating a diagnosis in a standardized, uniform manner, as the same classification and analysis techniques are applied to each slide. The results can then be reviewed by a pathologist in a manner that is faster and less prone to error. Accordingly, these automated techniques provide an improvement over manual techniques and are not subject to physician error.

The machine learning module may classify the cells into a respective cell category based upon morphological patterns of the cell. In an optional embodiment, the morphologic patterns may include cell shape, cell size, size of the nucleus, shape of the nucleus, granularity of the cytoplasm, or a fluorescent molecule that specifically binds to a marker on the surface of the cell. Any suitable feature may be identified as a morphological pattern. As different cells have different morphological patterns, the machine learning module may be trained to recognize different cell types in an automated manner by identifying one or more morphological patterns of the cell.

In an optional embodiment, a frequency of each cell type is determined, along with whether the frequency of each cell type is above or below a normal range for that cell type. Accordingly, present techniques allow for cell counts of respective cell categories to be determined in an automated manner and compared to respective control values. The number and type of cells may be used to generate a diagnosis or list of potential diagnosis for a patient. These techniques provide a uniform manner in which to identify a diagnosis or a plurality of candidate diagnoses, based on disease profiles that map cell counts and data analytics to particular diseases.

The present techniques may be extended to categorize cells into a variety of different categories. Optionally, the present techniques may identify subcategories of white blood cells, red blood cells, as well as other cells types, including cancer/tumor cells from solid tumors, malignant blood cells from myelodysplastic or tumoral processes as well as normal cells. Circulating cancer/tumor cells from solid tumors are typically present in low concentrations within a blood sample, and identification of a cancer/tumor cell may necessitate further diagnostic testing. However presence of aberrant numbers or morphologies of blood cells will allow the identification of hematological tumors (leukemias) as well as other non-tumoral diseases (sickle cell anemia, neutropenias, etc.).

Once the cells have been classified and analyzed to determine respective characteristics for each category, the information may be provided to the diagnostic module for a diagnosis. In an optional embodiment, a report may be generated including a diagnosis, a list of potential diagnoses, and a recommendation for additional testing when a definitive diagnosis cannot be made. In some cases, the system may provide a list of other diagnoses that may be confused with (misdiagnosed as) the diagnosis. Thus, these techniques may be used to enhance clinical judgement and reduce physician error. A plurality of diagnoses may be considered by the system to determine a diagnosis or list of candidate diagnoses for the patient, and reduce misdiagnosis of a condition.

In an optional embodiment, the system may be updated with current information, by using a natural language processing module to extract information from scientific and clinical literature to generate current disease profiles for the diagnosis module. This allows the system to stay up to date regarding changes in medical procedures and technology.

It is to be understood that the Summary is not intended to identify key or essential features of embodiments of the present disclosure, nor is it intended to be used to limit the scope of the present disclosure. Other features of the present disclosure will become easily comprehensible through the description below.

### BRIEF DESCRIPTION OF THE DRAWINGS

Generally, like reference numerals in the various figures are utilized to designate like components.

FIG. 1 is a block diagram of an example computing environment for automated blood pathology analysis, according to embodiments of the present disclosure.

FIG. 2 is a flowchart of an example blood pathology analysis workflow, beginning with obtaining the biological sample through providing diagnostic information, according to embodiments of the present disclosure.

FIG. 3 is a flow diagram showing generation of a training data set from scientific literature, according to embodiments of the present disclosure.

FIG. 4 is a flow diagram showing various inputs and outputs to the machine learning system, according to embodiments of the present disclosure.

FIG. 5 is a flow diagram showing various inputs and outputs of the diagnosis module, according to embodiments of the present disclosure.

FIG. 6 is a high level flow diagram of the blood pathology analysis system, according to embodiments of the present disclosure.

FIG. 7 are representations of microscopic images of blood stains that may be provided as input to train the blood pathology analysis system, according to embodiments of the present disclosure.

### DETAILED DESCRIPTION

Methods, systems, and computer readable media are provided for analyzing images from blood smears using machine learning and data analytics to diagnose a patient. Present techniques allow for automated and consistent classification and analysis of cells of a blood sample to generate a diagnosis. These techniques use a machine learning module to classify cells, based on morphological characteristics of the cells and in some cases, the presence of a probe that identifies a nucleotide mutation (e.g., detectable by FISH) or the presence of a cell marker to identify the cell (e.g., detectable by fluorophore or fluorochrome-conjugated antibodies). As additional biological samples are processed, the machine learning module may be trained using this data, and classification of cells may improve over time. These techniques can be extended to a variety of cell types, including but not limited to basophils, eosinophils, neutrophils, red blood cells (erythrocytes), macrophages, mast cells, myeloid derived suppressor cells (MDSCs), megakaryocyte cells, mesenchymal stem cells, monocytes, myeloid dendritic cells, B-cells, naive T cells, natural killer (NK) cells, plasmacytoid dendritic cells, platelets, T helper and T regulatory, as well as endothelial and epithelial cells, captured in the sample. Once the cells are classified, data analytics may be used to quantify aspects of the cell populations of each category.

Using automated techniques provides a robust approach to analyzing blood samples in a uniform, consistent manner. Additionally, the system may generate a personalized report for the physician based on the results of the classification and data analytics. The report may include a potential diagnosis, in cases in which the classification and data analytics identify a single disease. In other cases, the cell classification and data analytics may be associated with multiple candidate diagnoses, and the system may list the candidate diagnoses ranked based on likelihood and along with recommended follow-up testing to differentiate between these diseases. In some cases, the system may provide a list of other diagnoses that may be misdiagnosed or confused with the diagnosis or the list of diagnoses. These techniques aid a physician's decision making and diagnosis, highlighting frequently confused or closely similar diagnoses, and indicating the importance of performing differential diagnosis specific tests as well as reducing physician error and improving patient care.

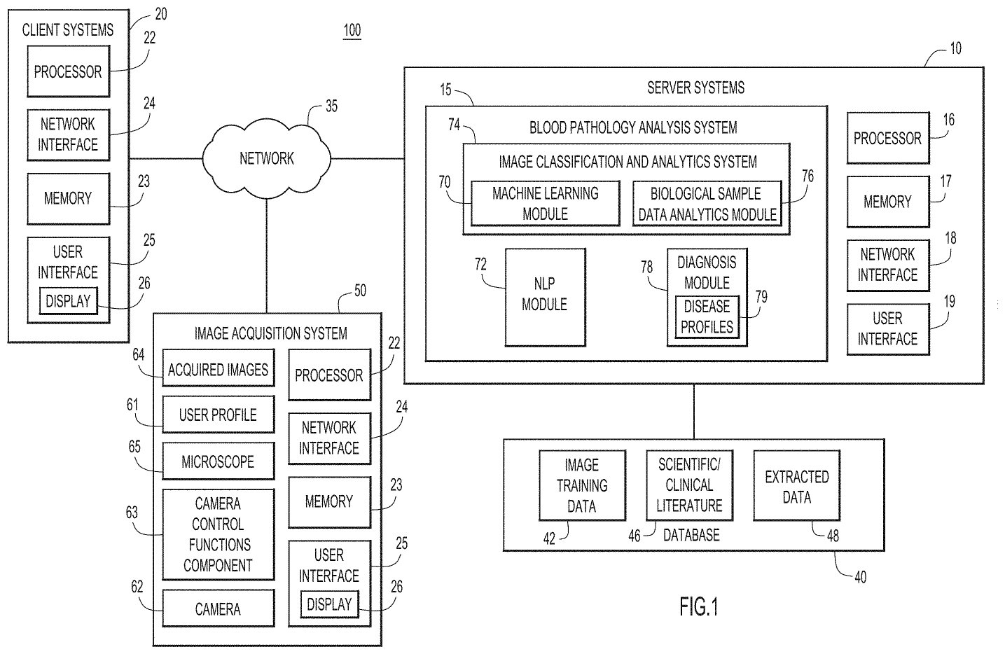

An example environment for use with present invention embodiments is illustrated in FIG. 1. Specifically, the environment includes one or more server systems 10, and one or more client or end-user systems 20. Server systems 10 and client systems 20 may be remote from each other and communicate over a network 35. The network may be implemented by any number of any suitable communications media (e.g., wide area network (WAN), local area network (LAN), Internet, Intranet, etc.). Alternatively, server systems 10 and client systems 20 may be local to each other, and communicate via any appropriate local communication medium (e.g., local area network (LAN), hardwire, wireless link, Intranet, etc.).

Client systems 20 enable users to submit documents (e.g., images, stained images, labeled images, etc.) to server systems 10 for analysis, to generate a diagnosis or a list of possible diagnoses. The server systems include a blood pathology analysis system 15 to analyze biological samples such as blood samples.

A database system 40 may store various information for the analysis (e.g., image training data 42, scientific/clinical literature 46, extracted data 48, etc.). The database system may be implemented by any conventional or other database or storage unit, may be local to or remote from server systems 10 and client systems 20, and may communicate via any appropriate communication medium (e.g., local area network (LAN), wide area network (WAN), Internet, hardwire, wireless link, Intranet, etc.). The client systems may present a graphical user (e.g., GUI, etc.) or other interface (e.g., command line prompts, menu screens, etc.) to solicit information from users pertaining to the desired documents (e.g., images of patient biological samples) and analysis, and may provide reports including analysis results (e.g., cell type and frequency analysis, diagnosis, list of possible diagnoses, follow-up diagnostic testing, misdiagnosis, etc.).

Server systems 10 and client systems 20 may be implemented by any conventional or other computer systems preferably equipped with a display or monitor, a base (e.g., including at least one processor 16, 22, one or more memories 17, 23 and/or internal or external network interfaces or communications devices 18, 24 (e.g., modem, network cards, etc.)), optional input devices (e.g., a keyboard, mouse or other input device), user interface 19, 25 (with a display 26), and any commercially available and custom software (e.g., server/communications software, blood pathology analysis system 15, browser/interface software, etc.).

Alternatively, one or more client systems 20 may analyze documents (e.g., images, scientific literature, etc.) to determine classification and analysis results of a blood sample by the blood pathology analysis system 15 when operating as a stand-alone unit. In a stand-alone mode of operation, the client system stores or has access to the data (e.g., image training data 42, scientific/clinical literature 46, extracted data 48, etc.), and includes a blood pathology analysis system 15. The graphical user (e.g., GUI, etc.) or other interface (e.g., command line prompts, menu screens, etc.) solicits information from a corresponding user pertaining to the desired documents and analysis, and may provide reports including analysis results (e.g., cell type and frequency analysis, diagnosis, list of possible diagnoses, follow-up diagnostic testing, misdiagnosis, etc.).

Blood pathology analysis system 15 may include one or more modules or units to perform the various functions of present invention embodiments described below. The various modules (e.g., natural language processing (NLP) module 72, image classification and analytics system 74, diagnosis module 78, etc.) may be implemented by any combination of any quantity of software and/or hardware modules or units, and may reside within memory 17 of the server and/or client systems for execution by processor 16, 22.

The blood pathology analysis system 15 may contain an image classification and analytics system 74, which classifies and analyzes individual cells of populations of cells of microscopic images. In some aspects, cells may be stained using histological methods known in the art (e.g., hematoxylin and eosin (H&E stain), PAS, toluidine blue, etc.) or labeled with fluorescently-labeled or enzymatically-conjugated antibodies, or any other suitable identification technique. Image classification and analytics system 74 may comprise a machine learning (ML) module 70, which classifies cells of an image into respective categories of cells based on morphological properties. For example, a cell may be classified as a red blood cell, a particular type of white blood cell, a cancer cell, a normal cell, etc. based on its appearance. The cells may or may not be labeled, stained, etc. In some cases, if the image contains damaged or lysed cells due to processing of the cells, the system may identify and filter out damaged or lysed cells so as not to skew the analysis. The machine learning model 70 may be trained using images of red blood cells (e.g., diseased and normal), particular types of white blood cells (e.g., diseased and normal) as well as using images of cancerous cells, normal cells, or any other suitable cell type. It is noted that blood samples may contain small amounts of circulating tumor cells or normal cells that may be identified by the system.

A variety of machine learning techniques may be implemented by ML module 70 including but not limited to statistical classification, supervised learning, unsupervised learning, artificial neural networks, deep learning neural networks, cluster analysis, random forest, dimensionality reduction, binary classification, decision tree, etc. Any suitable machine learning technique may be implemented by the machine learning module 70.

Classification of cells may be determined based on various morphological properties, including but not limited to cell shape, cell size, nucleus shape, nucleus size, nucleus to cell size ratio, intensity of stain or fluorescent signal, etc.

NPL module 72 is a natural language processing module which extracts various types of information from scientific/clinical literature 46, including but not limited to images of red blood cells, images of various types of white blood cells, images of normal cells, images of diseased cells, images of cancerous cells as well as morphological features of these respective types of cells (e.g., cell size, cell shape, cell radius, cell appearance, nucleus size, nucleus shape, nucleus radius, nucleus appearance, cell to nucleus ratio, cell diameter, cytoplasm granularity, chromosomal characteristics including the shape and density of chromosomes, intensity of staining, etc.) and information which links particular diseases to characteristics of these types of cells (e.g., cell counts, cell appearance, etc.). From this information, an image training data set 42 can be generated to train the machine learning module 70 to classify images of cells into a respective cell category. In some cases, the image training data 42 may be labeled by a subject matter expert.

NLP module 72 may extract information from scientific/clinical literature 46, which may include but is not limited to research publications, clinical trial information, laboratory procedures and protocols, review articles, pathology guides, or any other scientific or pathological hematological information that may be analyzed by NLP module 72. Extracted information may be stored as extracted data 48 and provided to diagnosis module 78 to provide a diagnosis of a disease.

Biological sample data analytics module 76 may obtain results from the machine learning module 70, and may perform various data analytics on the classified cell images. For example, biological sample data analytics module 76 may determine counts of each cell category to determine the number and type of cells in each image. This information may be provided to diagnosis module 78. Aberrant morphologies may also be identified by the system, e.g., morphologies that the machine learning module 70 is not able to classify. This information may be provided to the user for manual review.

Diagnosis module 78 may receive information from image classification and analytics system 74. The information from image classification and analytics system 74 may match one or more disease profiles 79 of diagnosis module 78, which allows a diagnosis to be made for a patient. Diagnosis module 78 may generate disease profiles based on cell characteristics (e.g., type of cell, cell counts, cell appearance, nucleus appearance, presence of cell markers, etc.) extracted from the literature by NLP module 72. The biological sample data from the image classification and analytics system may be compared to the disease profiles, to identify a diagnosis or candidate diagnoses based on classification of cells and data analytics. In other aspects, diagnosis module 78 may identify a plurality of diagnoses, if a definitive diagnosis cannot be determined. In general, any suitable characteristic may be used to generate a disease profile, including an increase or a decrease in particular types of cells, the appearance of a cell specific surface marker, as well as morphological changes of the cell, etc.

For example, if the biological sample is a blood sample, the system may identify sickle cells from normal red bloods. Red blood cells have a characteristic discoid shape, whereas sickle cells have a crescent or needle shape. Based on this morphological characteristic and other characteristics such as counts of sickle cells relative to normal blood cells, a diagnosis may be made.

As another example, the diagnosis module 78 may associate increases or decreases in particular types of white blood cells and/or red blood cells with a particular disease. For example, if the patient has high numbers of immature lymphocytes and monocytes, then a diagnosis of leukemia may be made.

Image acquisition system 50 may comprise a user profile 61, a microscope 65, a camera 62, camera control functions component 63, and acquired images 64 as well as a processor 22, a network interface 24, memory 23, a user interface 25, and a display 26. The user profile 61 may identify the operator of the image acquisition system 50. The camera 62 may acquire microscopic images (e.g., bright field, DIC, epifluorescent, etc.) of the cells, using the microscope, wherein the cells are typically fixed on slides. Camera control functions component 63 may be used to determine the settings for the camera 62 for image acquisition. Acquired images 64 may be provided to the blood pathology analysis system 15 for analysis.

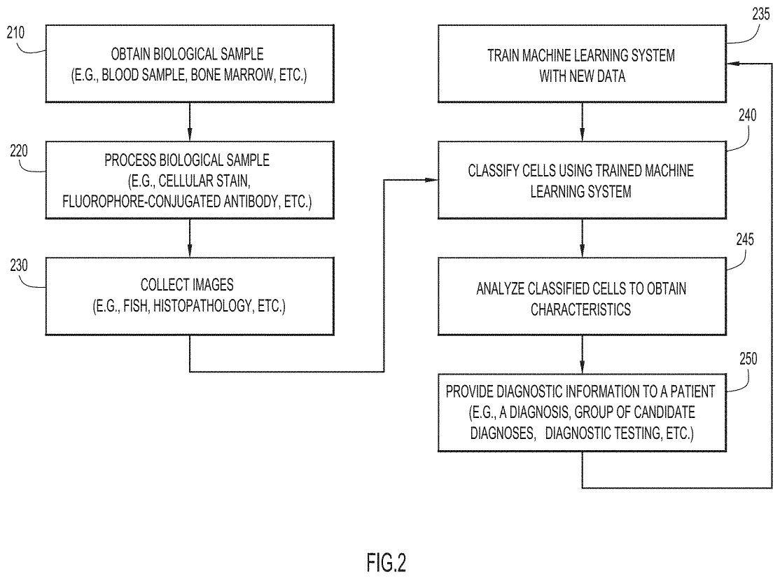

FIG. 2 shows an example flow chart beginning with obtaining a biological sample, through analysis by a machine learning system, and concluding with providing diagnostic information to a patient. Present techniques may be used to analyze blood smears (stained with any suitable reagent(s)), bone marrow aspirates, microscopic images (from fluorescent in situ hybridization (FISH), fluorescent-labeled or enzyme-conjugated antibodies, etc.) to provide a potential diagnosis (if a disease is present) based on the cell type and counts (e.g., total and relative cell counts) of red and white blood cell types, cell morphology (e.g., size, color, shape, nucleus shape, nucleus size, etc.), and data analytics. This data may be combined with previous biological sample data to generate real-world normal count range estimates. This process is described in additional detail as follows.

At operation 210, a biological sample is obtained from a patient. In some aspects, the biological sample may be a blood sample, a bone marrow sample, a tissue sample, or other sample that comprises heterogeneous cells. At operation 220, the biological sample is processed (e.g., stained, labeled) to allow determination of cell categories (types of cells). In some aspects, the cells may be fixed on a glass slide and subjected to a histological stain. The stain may allow visualization of the shape of the cell, as well as granularity of the cell cytoplasm, and/or compactness/density of cellular DNA, etc. Cellular stains may include but are not limited to H&E, methyl green, methylene blue, pyronin G, toluidine blue, acid fuschin, aniline blue, eosin, orange G, periodic acid-schiff reaction (PAS), alcian blue, van Gieson, reticulin stain, azan, etc. Additional stains are known in the art and any suitable stain may be used. In other aspects, the biological sample may be labeled with a fluorophore or fluorochrome-conjugated antibody. The antibody may be visualized by exposing the cells to florescence and imaging with an epifluorescent microscope or through enzymatic techniques under IHC. In this embodiment, the antibody may specifically bind to cell surface markers that identify the type of red blood cell, white blood cell, or other types of cell.

In some aspects, monoclonal antibodies may be used to bind to the cell surface to identify the cell type, e.g., a CD3 antibody, a CD7 antibody, a CD5 antibody (for T lymphocytes lineage); a CD19 antibody, a CD22 antibody, a CD20 antibody, a CD10 antibody (for B lymphocytes lineage); a CD13 antibody, a CD14 antibody, a CD33 antibody (for myeloid subsets); a HLA-DR antibody, a CD45 antibody, etc. Any suitable monoclonal antibody or combination thereof may be used to identify a cell type. In some aspects, the monoclonal antibody may be incubated with a secondary antibody that specifically binds to the monoclonal antibody, wherein the secondary antibody is conjugated to an enzyme that processes a colorimetric substrate or is conjugated to a fluorescent molecule for identification.

In other aspects, chromosomal material may be specifically identified using FISH. Using FISH, fixed cells may be exposed to a process resulting in nucleic acid denaturation and hybridization with a nucleic acid probe complementary to a specific DNA sequence that is indicative of the presence of a disease (e.g., a DNA sequence having a particular chromosomal alteration, translocation, inversion, deletion etc.). Fluorophore-conjugated antibodies may then be added to bind to the nucleic acid probe allowing visualization using epifluorescent microscopy.

At operation 230, images may be obtained of the processed biological sample using a microscope. At operation 240, the images are provided to a trained machine learning system. The machine learning system may analyze the images to determine cell types and classify cells into respective categories. At operation 245, classified cells may be analyzed to determine characteristics, e.g., the quantity of each cell type. In some aspects, the machine learning system may be provided with inputs which describe the biological sample (e.g., a blood sample), as well as inputs which describe processing conditions of the biological sample. For example, if cells are stained with H&E, the machine learning system will understand that this stain may be used to identify different types of cells in a tissue (e.g., erythrocytes, some immune cells, muscle cells, etc. as well as the presence of necrotic or hypernucleated cells).

At operation 250, diagnosis information may be provided to the patient. The diagnosis system 78 of the blood pathology analysis system 15 may provide a diagnosis for the patient based upon the machine learning results and analysis. In some aspects, results of classification and image analysis by the image classification and analytics system 74 may be provided to diagnosis module 78 for generating a diagnosis or list of potential diagnoses based upon disease profiles 78. The results of system 74 may be compared to various disease profiles to reach a diagnosis. In the event that the diagnostic module is not able to provide a diagnosis with a high degree of certainty, then the diagnostic module may include a list of candidate diagnoses along with one or more diagnostic tests to identify the patient's disease and references to supporting information (research articles, diagnostic and pathology guides etc.).

At operation 235, the machine learning system may be trained with training data 42, in some cases, data that has undergone curation by a subject matter expert. As new data (e.g., images that have been classified at operation 240) becomes available, then the machine learning system may be improved by training with this newly available data, at operation 235.

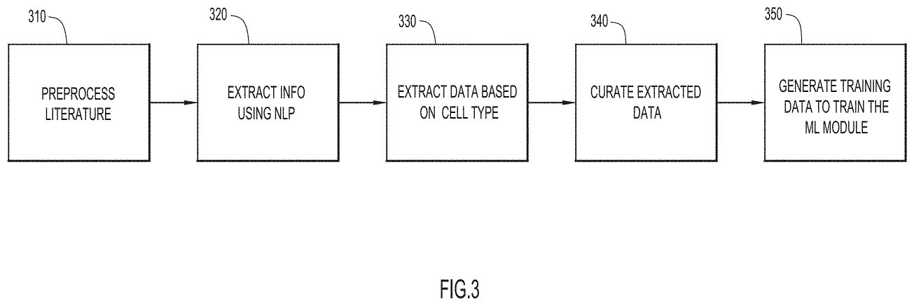

FIG. 3 shows a flow chart for generation of training data for the machine learning module 70. At operation 310, literature is pre-processed to be machine readable (e.g., by optical character recognition, etc.). The literature may include but is not limited to scientific publications, clinical reports, internal company information, experimental protocols, abstracts, conference proceedings, etc. At operation 320, NLP module 72 may be used to extract information useful for identifying documents comprising images suitable for generating a training data set. These images may be associated with information that identifies white blood cells (including monocytes, lymphocytes, basophils, eosinophils, granulocytes, etc.), red blood cells, diseased cells from the blood (e.g., lymphoblasts, cells with aberrant morphologies), and other cell types that may be found in small amounts circulating in the bloodstream (e.g., cancer cells, epithelial cells, etc.). At operation 330, the extracted information and corresponding documents may be grouped into categories corresponding to different cell types. At operation 340, the documents may be analyzed and images corresponding to the respective cell type may be extracted. Images may be curated and/or annotated prior to providing to the machine learning module. For example, the extracted data may be curated by a subject matter expert, such as a pathologist or other medical professional who has expertise in interpreting pathology and histology images. The curated and/or annotated data may be provided to the machine learning module as training data. In some aspects, the training data may include an image and corresponding information including the type of stain or label used to visualize the cell, the location of the cell within the image, the cell type, etc. In some aspects, features of the identified cells (e.g., size, shape, granularity, appearance of nucleus, etc.) may be used to classify cells of non-training data. At operation 350, the training data is provided to the blood pathology analysis system 15 to train the machine learning module to identify cell types for a particular biological sample corresponding to a patient.

Regarding staining, different white blood cell types may stain differently due to their intrinsic characteristics. For example, neutrophils may be stained mildly by acidic or basic stains. In contrast, granules of eosinophils may stain with acidic stains, while basophils may stain with basic stains. These features may also be used to identify cells by machine learning module 70.

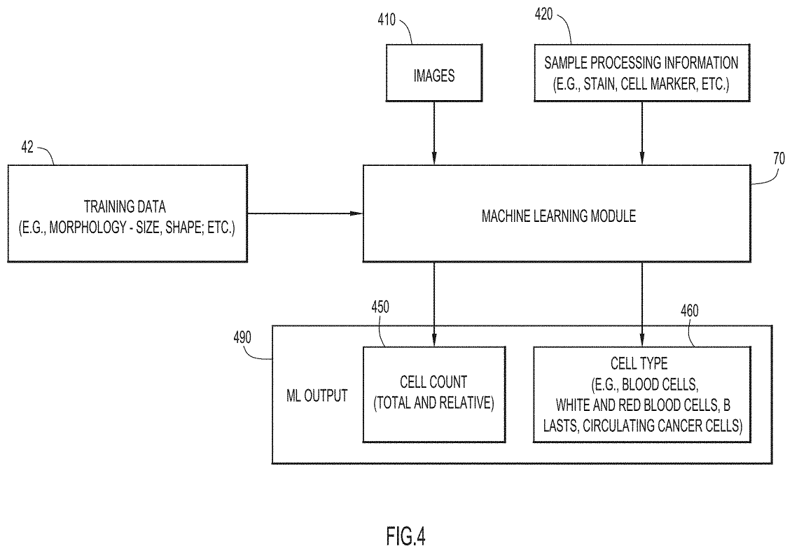

FIG. 4 shows a flowchart of inputs and outputs to machine learning module 70. As previously discussed, training data 42 may be provided to the machine learning module in order to train the machine learning module to identify cell types from a biological sample, such as a blood sample. Once the machine learning module is trained, it may analyze images of other biological samples (e.g., biological samples from patients) in order to provide a diagnosis for a patient. Images may be provided to the machine learning module 70 along with sample processing information 420 associated with the biological sample. The processing information may include but is not limited to the type of stain or fluorescent label used to visualize the cell, the type of chromosomal stains or probes used to visualize the nucleus or chromosomes, or other processing conditions associated with fixing and staining the cells. The machine learning module may analyze images of the processed biological sample to identify different cell types within the image. For example, a blood sample may comprise a variety of cell types, including but not limited to normal red blood cells, abnormal red blood cells (e.g., from sickle cell anemia), as well as white blood cells (including granulocytes/polymorphonuclear leukocytes (e.g., neutrophils, eosinophils, basophils, etc.), and mononuclear leukocytes (lymphocytes, monocytes, etc.)), and other cells (e.g., circulating cancer/tumor cells, epithelial cells, etc.).

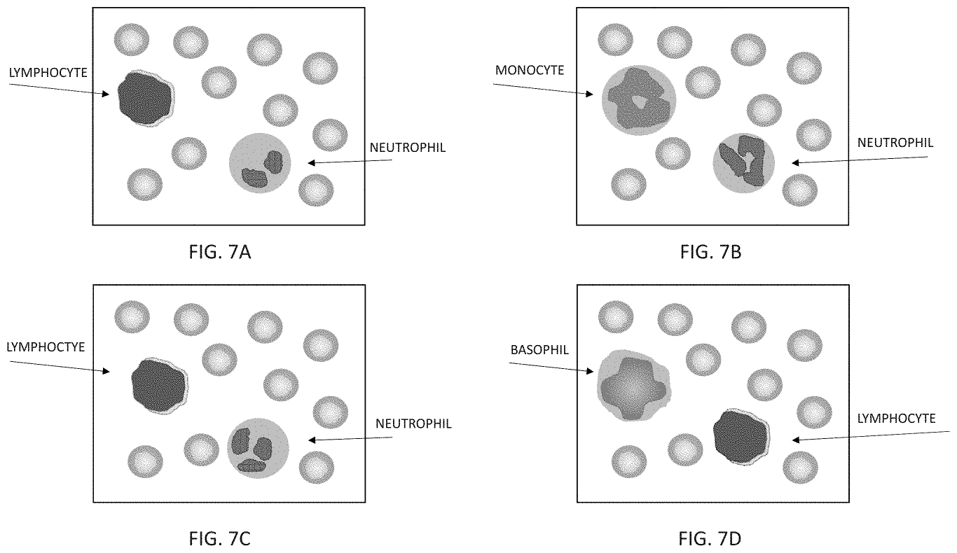

Cells may be identified based upon morphological properties. For example, normal red blood cells have a characteristic appearance in that these cells are anucleate with a characteristic discoid shape. In contrast, white blood cells include a nuclei and have other distinct morphological characteristics that allow for their identification. For example, characteristics of neutrophils may include about a 12 μm diameter, with a long, gated nuclei that may be separated into distinct lobes. Monocytes may have stained nuclei with a kidney bean or horseshoe shape. Lymphocytes may be about 7 to 9 μm in diameter with a round heterochromatic nucleus surrounded by a thin cover of cytoplasm (see also, FIGS. 7A-7D).

Once the images are analyzed by the machine learning module 70, the machine learning module may provide various types of outputs. The machine learning output 490 may include but is not limited to cell count (e.g., total number of cells and relative number of cells per cell type). For example, cell counts may include the number of normal red blood cells, abnormal red blood cells, and the number of different types of white blood cells as well as other types of cells identified in the sample. In some aspects, cell counts may vary based on other factors including ethnicity, geographical location, and diet. These parameters may be tracked by the system as well.

Cell classification by a machine learning (ML) module may be based on a combination of morphological features and cellular markers, allowing multiple types of cells to be identified. In some aspects, the ML module may analyze multiple types of assays (e.g., fluorescence in situ hybridization (FISH) images, histological staining, fluorophore-conjugated antibody labeling, etc.) from the same patient. The system may combine results from blood smears, bone marrow samples, etc. during processing.

In some aspects, a blood sample is placed on a slide and provided to a microscope to generate one or more microscopic images of the blood sample, optionally at various magnifications. The images may be analyzed by image classification and analytics system 74 to classify different types of blood cells into respective categories (e.g., using machine learning based on morphological features, stains, labels, etc.), and to determine the frequency of each cell type. Cells that do not match a normal blood cell type may be identified as well.

As an example, blood tumors may have higher than expected frequencies of a specific blood cell type (e.g. lymphocyte, monocytes, platelets, etc.) as well as more complex patterns including a combination of higher numbers of certain cell subtypes together with lower numbers of a different subtype. The morphology of various types of blood cells are known in the art, and the machine learning module may be trained with images available from the medical literature and scientific literature. Aberrant frequencies of cell types may be utilized to determine a diagnosis. The blood pathology analysis system 15 may be integrated with existing microscopy software, developed as client-server software, or provided as stand-alone software.

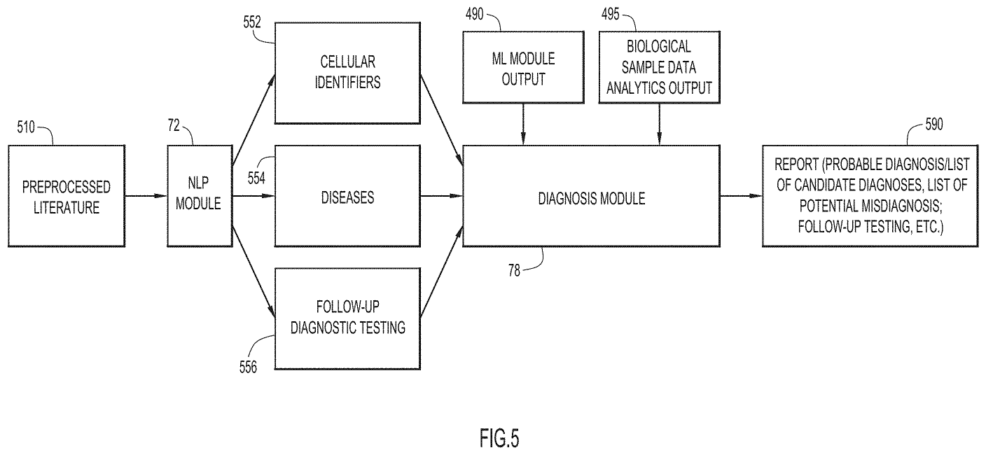

FIG. 5 is a flowchart showing extraction of literature information that may be provided to diagnosis module 78, to generate disease profiles 79 as well as a report for the patient regarding a diagnosis, a list of candidate diagnoses, a list of potential misdiagnoses, as well as information regarding follow-up testing when a diagnosis cannot be determined. Preprocessed literature 510, which may be processed by performing optical character recognition on the text to render the documents machine readable, may be provided to NLP module 72, which extracts various types of information from the literature including cellular identifiers 552, diseases 554, follow-up diagnostic testing 556, etc. Cellular identifiers may include any type of information used to identify a cell, e.g., one or more cell surface proteins that may be detected by respective antibodies or other suitable reagents, one or more intracellular markers that may be detected by respective antibodies or other suitable reagents, morphologies of cell types, etc. For example, common extracellular identifiers for neutrophils may include CD10, CD11b, CD11c, CD13, CD14, CD15 (SSEA-1), CD16/32, CD31, CD33, CD62L, CD64, CD66b, CD88, CD114 (G-CSFR), CXCR1, CXCR2, GR-1, JAML, TLR2. Common extracellular identifiers for monocytes may include CCR2, CD11b, CD11c, CD14, CD16, CD43, CD86, CD115, CD172a (SIRPα), CD209 (DC-SIGN), CX3CR1, F4/80, HLA-DR, Ly6C, and MHC Class II. Many different types of markers are known for different types of cells. Diseases 554 include diseases associated with the cellular identifiers as well as counts of respective cell types displaying the cellular identifiers. For example, cells displaying certain markers above (overexpression) or below (under expression) a control or reference cell displaying a normal range of the cellular marker may be indicative of a disease. In other cases, the number and types of cells within a biological sample may be indicative of a disease. By measuring the amounts and types of different cells in the blood sample, various medical conditions may be diagnosed.

In cases in which the system cannot reach a diagnosis with a high degree of confidence, the system may provide a list of follow-up diagnostic tests 556 to distinguish between different diseases. For example, high levels of neutrophils may be indicative of rheumatoid arthritis or Hodgkin's lymphoma. Eosinophils may be associated with allergic conditions or parasitic infections. High levels of monocytes may be correlated with lupus, cancer, and/or other blood proliferative diseases such as myelomonocytic leukemia or monocytic leukemia. Accordingly, follow-up diagnostic testing may include additional diagnostic tests to distinguish between different medical conditions. Additionally, the system may provide a list of misdiagnoses that are frequently confused with the diagnosis provided by the system.

The output of the image classification and analytics system may provide information from the ML module output 490 along with data analytics 495 including cell types and cell counts, and a list of cellular markers and morphological characteristics associated with the blood sample under analysis. This information may be provided to the diagnosis module 78, where it may be matched to disease profiles generated by the diagnosis module. In some aspects, a disease profile may include characteristics of cells associated with a disease. For example, a disease profile for acute leukemia may include a percentage of blasts (undifferentiated blood cells) over 30% of the cells of the biological (in this case blood) sample. To distinguish between acute myeloid leukemia (AML) and acute lymphoid leukemia (ALL), markers may be evaluated. For example, the presence of CD3, CD5, CD13, CD14, CD19, CD45, HLA-DR, and TdT may indicate AML. A different set of markers may indicate the presence of ALL.

The diagnosis module 78 identifies one or more disease profiles 79, which match the information provided by the system 74 regarding the biological sample. A report 590 is generated and provided to the patient regarding one or more of a diagnosis, a list of candidate diagnoses, a list of potential misdiagnoses, as well as recommended follow-up testing. Present techniques may also, for related and/or frequently confused diagnoses (misdiagnoses), suggest additional tests for differential diagnosis of the disease. This information may be integrated with the information from biological sample analysis, including cell counts, and microscopic images of biological samples, such as FISH or H&E stained histologic slides.

In other aspects, information from analysis of the biological sample may be combined with other parameters (e.g., such as age, ethnicity, etc.) to provide candidate diagnoses and/or suggestions of additional diagnostic tests that differentiate between the candidate diagnoses. These techniques may accumulate knowledge from previous sample classification and analysis to adjust and generate more accurate ranges of normal and/or disease parameters.

The diagnosis module 78 may evaluate different types of information, such as cell counts, cell types, presence of markers, morphological properties, etc. when determining a potential diagnosis. If the information provided by the ML module 490 and biological sample data analytics output 495 is definitively associated with a diagnosis, then the system will generate a diagnosis for the patient. For instance, certain diseases may be definitively associated with particular morphologies, the presence of specific markers or combinations of markers, and/or cell counts. In other cases, the information provided by the ML module 490 and biological sample data analytics output 495 may not be sufficient to make a diagnosis, and the system may list a plurality of candidate diagnoses and indicate that additional information (e.g., further testing) is needed.

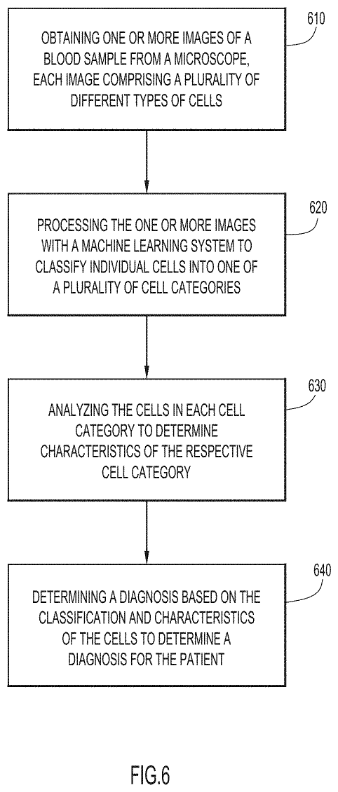

FIG. 6 is a high level flowchart of operations for the blood pathology system. At operation 610, one or more images of a blood sample from a microscope are obtained, each image comprising a plurality of different types of cells. At operation 620, the one or more images are processed by a machine learning system to classify individual cells into one of a plurality of cell categories. At operation 630, the cells in each cell category are analyzed to determine characteristics of the respective cell category. At operation 640, a diagnosis for the patient based on the classification (e.g., cell types) and characteristics (e.g., cell counts, expression level of markers, etc.) of the cells is determined.

FIGS. 7A-7D show various representations of images of white and red blood cells. In this example representation, the cells have been stained using known techniques for visualization. FIG. 7A shows an example of a neutrophil and a lymphocyte in a field of red blood cells. As shown by this example, lymphocytes have a distinct morphological appearance as compared to neutrophils. In particular, lymphocytes display a large nucleus that nearly spans the diameter of the cell image. In contrast, a neutrophil has a lobed, condensed nucleus that takes up less than about half of the cell image. FIG. 7B shows a representative image of a neutrophil and a monocyte in a field of red blood cells. The monocyte nucleus is more diffuse that the neutrophil nucleus, and has a characteristic stain that is more diffuse than the neutrophil nucleus. FIG. 7C and FIG. 7D show additional examples of representative training images of neutrophils and lymphocytes for the blood pathology system. In FIG. 7D, a representative image of a basophil is provided.

Present techniques may be applied to any biological sample in which staining and/or processing allows resolution of a cell boundary and/or internal structures of a cell, and in which the presence and amount of various cell types may be associated with a disease and/or categories of diseases.

In some aspects, the system may classify and analyze cells. In other aspects, the system may classify and analyze cells to generate a diagnosis for a patient. In other aspects, the system may classify and analyze cells to generate a list of candidate diagnoses for a patient, and optionally, a list of follow-up diagnostic tests to distinguish between different candidate diagnoses. For example, a ML process may be used to analyze pathology slides for making a diagnosis of hematological or other malignancies in an automated manner.

Advantages of present techniques include fast automated detection, classification and quantification of different types of cells in biological samples, such as blood smears, tissue samples, and other microscopic images, to diagnose diseases. In some cases, present techniques can diagnose complex cases, which may be difficult for a pathologist to identify from a manual review of the slides. The present techniques may improve the speed and accuracy of diagnoses as compared to manual review by a pathologist, as analysis and processing are not subject to user bias. Additionally, as more samples are analyzed and processed, this new data may be included in the training data set for the machine learning system to improve the accuracy and precision of the blood pathology analyzer.

Advantages of present techniques include improving patient care, e.g., by obtaining a faster, more accurate diagnosis, allowing patients to start appropriate treatment in a time efficient manner. Additionally, clinicians would be prompted to perform additional tests if a diagnosis is not definitively made, leading to improved patient care. A robust expertly trained ML module for pathology review and diagnosis of hematological malignancies may not only speed the diagnosis of patients but also generate standardized and reproducible diagnostic reports to support a physician's decision making and prevent potential bias between different pathologists.

Present techniques incorporate data from scientific literature and diagnostic methods through NPL to provide information on currently approved and investigational treatments matching the diagnosis. In some cases, potentially related diseases are provided to the patient, and additional tests may be recommended for differential diagnosis.

It will be appreciated that the embodiments described above and illustrated in the drawings represent only a few of the many ways of implementing embodiments for automating diagnosis of a disease based on a biological sample from a patient. In cases in which the machine learning system cannot reach a suitable diagnosis, the images and quantitative results may be tagged by the machine for review by a pathologist.

Any suitable cell may be analyzed by these techniques, including but not limited to astrocytes, basophils, B cells, embryonic stem cells, endothelial cells, eosinophils, epithelial cells, erythrocytes, fibroblasts, hematopoietic stem cells, macrophages, mast cells, MDSC cells, megakaryocyte cells, mesenchymal stem cells, microglia, monocytes, myeloid dendritic cells, naive T cells, neurons, neutrophils, NK cells, plasmacytoid dendritic cells, platelets, stromal cells, T helper and T regulatory cells, etc.

The environment of the present invention embodiments may include any number of computer or other processing systems (e.g., client or end-user systems, server systems, etc.) and databases or other repositories arranged in any desired fashion, where the present invention embodiments may be applied to any desired type of computing environment (e.g., cloud computing, client-server, network computing, mainframe, stand-alone systems, etc.). The computer or other processing systems employed by the present invention embodiments may be implemented by any number of any personal or other type of computer or processing system (e.g., desktop, laptop, PDA, mobile devices, etc.), and may include any commercially available operating system and any combination of commercially available and custom software (e.g., browser software, communications software, server software, blood pathology analysis system 15, image acquisition system 50, etc.). These systems may include any types of monitors and input devices (e.g., keyboard, mouse, voice recognition, etc.) to enter and/or view information.

It is to be understood that the software (e.g., blood pathology analysis system 15, including image classification and analytics system 74, NLP module 72, and diagnosis module 78, etc.) of the present invention embodiments may be implemented in any desired computer language and could be developed by one of ordinary skill in the computer arts based on the functional descriptions contained in the specification and flow charts illustrated in the drawings. Further, any references herein of software performing various functions generally refer to computer systems or processors performing those functions under software control. The computer systems of the present invention embodiments may alternatively be implemented by any type of hardware and/or other processing circuitry.

The various functions of the computer or other processing systems may be distributed in any manner among any number of software and/or hardware modules or units, processing or computer systems and/or circuitry, where the computer or processing systems may be disposed locally or remotely of each other and communicate via any suitable communications medium (e.g., LAN, WAN, Intranet, Internet, hardwire, modem connection, wireless, etc.). For example, the functions of the present invention embodiments may be distributed in any manner among the various end-user/client and server systems, and/or any other intermediary processing devices. The software and/or algorithms described above and illustrated in the flow charts may be modified in any manner that accomplishes the functions described herein. In addition, the functions in the flow charts or description may be performed in any order that accomplishes a desired operation.

The software of the present invention embodiments (e.g., blood pathology analysis system 15, including image classification and analytics system 74, NLP module 72, and diagnosis module 78, etc.) may be available on a non-transitory computer useable medium (e.g., magnetic or optical mediums, magneto-optic mediums, floppy diskettes, CD-ROM, DVD, memory devices, etc.) of a stationary or portable program product apparatus or device for use with stand-alone systems or systems connected by a network or other communications medium.

The communication network may be implemented by any number of any type of communications network (e.g., LAN, WAN, Internet, Intranet, VPN, etc.). The computer or other processing systems of the present invention embodiments may include any conventional or other communications devices to communicate over the network via any conventional or other protocols. The computer or other processing systems may utilize any type of connection (e.g., wired, wireless, etc.) for access to the network. Local communication media may be implemented by any suitable communication media (e.g., local area network (LAN), hardwire, wireless link, Intranet, etc.).

The system may employ any number of any conventional or other databases, data stores or storage structures (e.g., files, databases, data structures, data or other repositories, etc.) to store information (e.g., acquired images 64, image training data 42, scientific/clinical literature 46, extracted data 48, image classification from the machine learning module 70, cell counts and other properties from the biological sample data analytics module 76, reports from the diagnosis module, etc.). The database system may be implemented by any number of any conventional or other databases, data stores or storage structures (e.g., files, databases, data structures, data or other repositories, etc.) to store information (e.g., acquired images 64, image training data 42, scientific/clinical literature 46, extracted data 48, image classification from the machine learning module 70, cell counts and other properties from the biological sample data analytics module 76, reports from the diagnosis module, etc.). The database system may be included within or coupled to the server and/or client systems. The database systems and/or storage structures may be remote from or local to the computer or other processing systems, and may store any desired data (e.g., acquired images 64, image training data 42, scientific/clinical literature 46, extracted data 48, image classification from the machine learning module 70, cell counts and other properties from the biological sample data analytics module 76, reports from the diagnosis module, etc.).

The present invention embodiments may employ any number of any type of user interface (e.g., Graphical User Interface (GUI), command-line, prompt, etc.) for obtaining or providing information (e.g., acquired images 64, image training data 42, scientific/clinical literature 46, extracted data 48, image classification from the machine learning module 70, cell counts and other properties from the biological sample data analytics module 76, reports from the diagnosis module, etc.), where the interface may include any information arranged in any fashion. The interface may include any number of any types of input or actuation mechanisms (e.g., buttons, icons, fields, boxes, links, etc.) disposed at any locations to enter/display information and initiate desired actions via any suitable input devices (e.g., mouse, keyboard, etc.). The interface screens may include any suitable actuators (e.g., links, tabs, etc.) to navigate between the screens in any fashion.

The report may include any information arranged in any fashion, and may be configurable based on rules or other criteria to provide desired information to a user (e.g., a definitive diagnosis, possible candidate diagnoses, list of similar diseases (common misdiagnoses), follow-up medical testing, etc.).

The present invention embodiments are not limited to the specific tasks or algorithms described above, but may be utilized for any suitable biological sample in which morphology and cell type count may indicate the presence of a disease.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises”, “comprising”, “includes”, “including”, “has”, “have”, “having”, “with” and the like, when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

What is claimed is:

1. A computer-implemented method of processing microscopic images of a blood sample from a patient comprising:
obtaining one or more images of a blood sample from a microscope, each image comprising a plurality of different types of cells;
processing the one or more images with a machine learning module to classify individual cells into one of a plurality of cell categories;
analyzing the cells in each cell category to determine characteristics of the respective cell category; and
determining a potential diagnosis based on the classification and characteristics of the cells to determine a diagnosis for the patient.

2. The method of claim 1, wherein the machine learning module classifies the cells into a respective cell category based upon morphological patterns of the cell.

3. The method of claim 2, wherein the morphologic patterns include cell shape, cell size, size of the nucleus, shape of the nucleus, granularity of the cytoplasm, or a fluorescent marker that specifically binds to a marker on the surface of the cell.

4. The method of claim 1, wherein analyzing further comprises:
determining a frequency of each cell type; and
determining whether the frequency of each cell type is above or below a normal range for that cell type.

5. The method of claim 1, wherein the cell categories include any one or more of a red blood cell, a white blood cell, a cancer cell, or a normal cell.

6. The method of claim 5, wherein the white blood cell categories include any one or more of the following cell categories: leukocytes, monocytes, granulocytes, basophils, and eosinophils.

7. The method of claim 1, further comprising generating a report including a list of potential diagnoses and a recommendation for additional testing when a definitive diagnosis cannot be made.

8. The method of claim 1, wherein a natural language processing module extracts information from the scientific and clinical literature to generate disease profiles for the diagnosis module.

9. A system for processing microscopic images of a blood sample from a patient, the system comprising at least one processor configured to:
obtain one or more images of a blood sample from a microscope, each image comprising a plurality of different types of cells;
process the one or more images with a machine learning module to classify individual cells into one of a plurality of cell categories;
analyze the cells in each cell category to determine characteristics of the respective cell category; and
determine a diagnosis based on the classification and characteristics of the cells to determine a diagnosis for the patient.

10. The system of claim 9, wherein the machine learning module classifies the cells into a respective cell category based upon morphological patterns of the cell.

11. The system of claim 10, wherein the morphologic patterns include cell shape, cell size, size of the nucleus, shape of the nucleus, granularity of the cytoplasm, or a fluorescent marker that specifically binds to a marker on the surface of the cell.

12. The system of claim 9, wherein the processor is further configured to:
determine a frequency of each cell type; and
determine whether the frequency of each cell type is above or below a normal range for that cell type.

13. The system of claim 9, wherein the cell categories include any one or more of a red blood cell, a white blood cell, a cancer cell, or a normal cell, and wherein the white blood cell categories include any one or more of the following cell categories: erythrocytes, leukocytes, monocytes, granulocytes, basophils, and eosinophils.

14. The system of claim 9, wherein the processor is further configured to generate a report including a list of potential diagnoses and a recommendation for additional testing when a definitive diagnosis cannot be made.

15. The system of claim 9, wherein a natural language processing module extracts information from the scientific and clinical literature to generate disease profiles for the diagnosis module.

16. A computer program product for processing microscopic images of a blood sample from a patient, the computer program product comprising a computer readable storage medium having program instructions embodied therewith, the program instructions executable by a processor to:
obtain one or more images of a blood sample from a microscope, each image comprising a plurality of different types of cells;
process the one or more images with a machine learning module to classify individual cells into one of a plurality of cell categories;
analyze the cells in each cell category to determine characteristics of the respective cell category; and
determine a diagnosis based on the classification and characteristics of the cells to determine a diagnosis for the patient.

17. The computer program product of claim 16, wherein the machine learning module classifies the cells into a respective cell category based upon morphological patterns of the cell.

18. The computer program product of claim 17, wherein the morphologic patterns include cell shape, cell size, size of the nucleus, shape of the nucleus, granularity of the cytoplasm, or a fluorescent marker that specifically binds to a marker on the surface of the cell.

19. The computer program product of claim 16, wherein the computer readable program instructions are executable to:
determine a frequency of each cell type; and
determine whether the frequency of each cell type is above or below a normal range for that cell type.

20. The computer program product of claim 16, wherein the computer readable program instructions are executable to generate a report including a list of potential diagnoses and a recommendation for additional testing when a definitive diagnosis cannot be made.
